(19) United States
(12) Patent Application Publication
(10) Pub. No.: US 2011/0152697 A1
(43) Pub. Date: Jun. 23, 2011

(54) Circulatory Pressure Monitoring Using Infusion Pump Systems

(75) Inventor: Yasuhiro Kawamura, Tokyo (JP)

(73) Assignee: K&Y Corporation

(21) Appl. No.: 12/972,394

(22) Filed: Dec. 17, 2010

Related U.S. Application Data
(60) Provisional application No. 61/287,881, filed on Dec. 18, 2009.
(60) Provisional application No. 61/287,903, filed on Dec. 18, 2009.
(60) Provisional application No. 61/287,912, filed on Dec. 18, 2009.
(60) Provisional application No. 61/287,991, filed on Dec. 18, 2009.

(51) Int. Cl. A61B 5/021

(52) U.S. Cl. 600/485

(57) ABSTRACT

A low cost, transportable system for monitoring the central venous pressure of a patient receiving an infusion is provided. The pressure monitoring system of the present invention employs a pump and a flow meter in order to supply infusion fluids to a patient. Based upon the control factors and changes thereof communicated to the pump by a controller in order to achieve and maintain a desired infusion fluid flow rate, relative changes in patient's venous pressure and/or quantitative pressure data is obtained.

### RELATED APPLICATIONS

This application claims priority to U.S. Provisional Application Ser. No. 61/287,881 filed Dec. 18, 2009, entitled MEMS Pump for Medical Infusion Pump; U.S. Provisional Application Ser. No. 61/287,903 filed Dec. 18, 2009, entitled Pump Stay; U.S. Provisional Application Ser. No. 61/287,912 filed Dec. 18, 2009, entitled Micro Infusion Pump System Software; and U.S. Provisional Application Ser. No. 61/287,991 filed Dec. 18, 2009, entitled Central Venous Pressure Monitoring Using Micro Infusion Pump, the contents of which are each incorporated in their entirety herein.

### FIELD OF THE INVENTION

The present invention relates to circulatory pressure monitoring systems and related methods and, more particularly, to central venous pressure monitoring systems employing piezoelectric driven infusion pumps.

### BACKGROUND OF THE INVENTION

Information gained from a patient's body during medical treatment is an important tool in assessing a patient's health. In particular, biological information from patients can be used as an indicator of the state of a disease or medical treatment. Such information can typically only be obtained through the use of specialized medical equipment. However, such specialized medical equipment is often costly and/or impractical to move and use outside of a hospital, clinic, or other medical venue. Accordingly, it is extremely difficult to obtain real-time biological information for patients undergoing homecare or care at another remote location. In view of the trend towards the increasing use of home and other remote patient care models, it becomes apparent that the inability to obtain real-time patient information is a serious problem and, in some cases, may contribute to the development of serious health conditions in a patient.

Real-time central venous pressure is one example of biological information that can be crucial to a patient's care. For example, central venous hyperalimentation, a high calorie infusion method, is broadly practiced as a nutritional supplementation method during postoperative recovery. Because 2000 cc or more of liquids are infused into a patient's body in a day, particularly in the case of surgery involving the circulatory system, the central venous pressure is generally monitored during the infusion. Central venous pressure values of the patient serves to indicate whether or not a load is being placed on the patient's heart by, among other things, the relatively high-volume infusion. When high calorie infusions are performed, a double catheter (double lumen) or triple catheter (triple lumen) is often used. One of the lumens is used to introduce infusion fluid, and a second lumen is used to measure of central venous pressure. Pressure meters operable to measure the central venous pressure are significantly expensive. Hence, such meters are not available to patients receiving central venous hyperalimentation as part of the homecare model.

What is needed in the field is a low cost, transportable means for determining patient circulatory pressure.

### OBJECTS AND SUMMARY OF THE INVENTION

The present invention provides a low cost, transportable means for monitoring the central venous pressure of a patient receiving an infusion. The pressure monitoring system of the present invention preferably employs a piezoelectric micropump and a flow meter for infusion of fluids to a patient. Based upon the control factors and changes thereof communicated to the micropump in order to achieve and maintain a desired infusion fluid flow rate, relative changes in patient's venous pressure and/or quantitative pressure data is obtained.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is diagram of a pressure monitoring system according to one embodiment of the present invention.

FIG. 2 is diagram of a pressure monitoring system according to one embodiment of the present invention.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

The present invention provides a low cost, transportable, and highly accurate circulatory pressure monitoring system that can be used in a hospital or clinic but that is also suitable for use during patient homecare. Broadly speaking, the present invention achieves these goals by utilizing an infusion pump to provide the infusion fluid to the patient and closely monitoring the infusion pump control factors in order to derive patient circulatory pressure data.

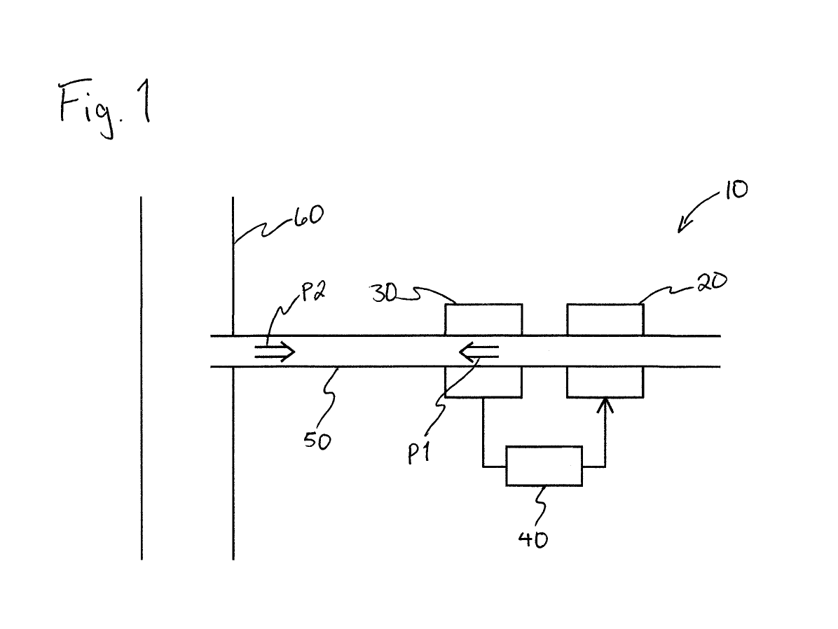

As shown in FIG. 1, pressure monitoring system 10 according to one embodiment of the present invention comprises an infusion pump 20, a flow meter 30, a controller 40, and a patient fluid line 50. The patient line 50 is in fluid communication with a patient vein 60 downstream of the pump 20 and a fluid reservoir, not shown, upstream of the pump 20.

With respect to the pump 20, it is contemplated that a variety of types of infusion pumps, including peristaltic pumps, syringe pumps, and elastomeric pumps, can be employed as the pump 20. In order to achieve the greatest accuracy and convenience, it is preferred that the pump 20 be a microelectromechanical, or MEMS, micropump driven by a piezoelectric effect. In brief, such micropumps can be fabricated using known integrated circuit fabrication methods and technologies. For example, using integrated circuit manufacturing fabrication techniques, small channels can be formed on the surface of silicon wafers. By attaching a thin piece of material, such as glass, on the surface of the processed silicon wafer, flow paths and fluid chambers can be formed from the channels and chambers. A layer of piezoelectric material, or a piezoelectric body such as quartz, is then attached to the glass on the side opposite the silicon wafer. When a voltage is applied to the piezoelectric body, a reverse piezoelectric effect, or vibration, is generated by the piezoelectric body and transmitted through the glass to the fluid in the chamber. In turn, a resonance is produced in the fluid in the chamber of the silicon wafer. Through the inclusions of valves and other design features in the fluid flow paths, a net directional flow of fluid through the chamber formed by the silicon wafer and the glass covering can be achieved. Examples of such pumps and related control systems are described in greater detail in the Assignee's copending U.S. Patent Application Nos. (TBD) entitled Infusion Pump and (TBD) entitled Patient Fluid Management System, the contents of which are herein incorporated in their entirety.

The flow meter 30 may comprise a variety of flow meters known in the field. For example, the flow meter 30 may be configured to determine fluid flow rates by employing a heater that heats the fluid being monitored and senses the flow of the heated fluid downstream of the heater. Such flow meters are available from Sensirion AG of Switzerland and Siargo Incorporated of the United States of America and are described in greater detail in at least U.S. Pat. No. 6,813,944 to Mayer et al. and U.S. Publication No. 2009/0164163, which are herein incorporated by reference. Alternatively, the flow meter 34 may be configured to employ two pressure sensors positioned on each side of a constriction within the fluid flow path. Fluid flow rates are determined by the relative difference between the pressure sensors and changes thereof.

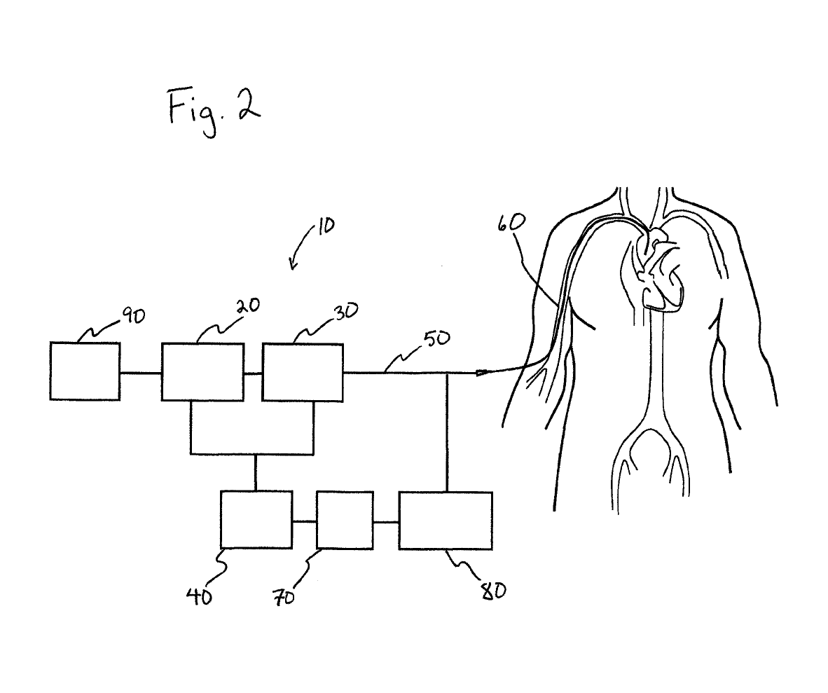

While the flow meter 30 is shown in FIGS. 1 and 2 as being separate from the pump 20 and the controller 40, it will be understood that the flow meter 30 may be physically incorporated into either the pump 20 or the controller 40, or both. The controller 40 is in electrical communication with the pump 20 and flow meter 30 and functions to provide power to each of these components and to receive any control or data feedback, e.g. fluid flow rate data, generated by the pump 20 and the flow meter 30. The patient line 50 may comprise a variety of different tubing and catheter systems known in the field.

In operation, the pump 20 generates an infusion pressure, indicated in FIG. 1 by the arrow P1, and thereby generates a net fluid flow through the patient line 50 into the patient vein 60 by overcoming the patient central venous pressure, indicated in FIG. 1 by the arrow P2. If the throughput of pump 20 is maintained constant and the patient venous pressure P2 remains constant, than the infusion pressure P1 minus the patient venous pressure P2, i.e. (P1-P2), will also remain constant. However, if the patient venous pressure P2 changes, the throughput of the pump 20 and, accordingly, the infusion pressure P1 generated by the pump 20, must be change in order to maintain the constant fluid flow rate to the patient. In order to achieve the necessary change in throughput of the pump 20, the control factors for the pump 20 are changed. For example, such control factors include, but are not limited to; the voltage and/or the frequency at which the voltage is provided to the pump 20 are changed as well as the rate at which the voltage is increased and decreased. Consequently, changes in the patient venous pressure P2 are directly reflected in the control factors provided to the pump 20 in order that the pump 20 generates or maintains the desire fluid flow to the patient. In other words, by monitoring changes in the control factor for the pump 20, it is possible to monitor and determine information regarding a patient's central venous pressure.

It will be recognized that, according to the above description, only relative changes in the patient venous pressure P2 are determined. In order to determine quantitative changes in the patient venous pressure P2, the changes in the control factors for the pump 20 must be calibrated or correlated with measured pressure values. However, once the control factors for the pump 20 are calibrated with measured data, it is possible to derive the venous pressure P2 of the patient based solely on readily available control factors for the pump 20, i.e. it is possible to determine the patient venous pressure P2 without expensive, non-transportable medical equipment such as blood pressure meters.

Quantitative venous pressure data is derived by experimentally correlating specific control factors for the pump 20 with specific pressures, such as central venous pressures, over a functional range of control factors and pressures. The pressure monitoring system 10 of the present invention can be initially calibrated relative to correlated control factor and pressures data by independently measuring a patient's central venous pressure and directly inputting the patient's central venous pressure into a user interface 70 shown in FIG. 2.

Alternatively, a more accurate initial calibration can be achieved by, at least temporarily, combining a blood pressure meter with pressure monitoring system 10. For example, with reference to the FIG. 2, before the patient line 50 is connected to the patient, the system 10 is primed by pumping the infusion fluid, such as, for example total parental nutrition, from a fluid reservoir 90 through the pump 20, the flow meter 30, and the patient line 50 while the patient line 50 is in an open or unconstructed configuration. As the infusion fluid is pumped through the pump 20, the flow meter 30, and the patient line 50, a user indicates to the controller 40 through the user interface 70 that this unconstructed flow represents a first calibration point in a zero back pressure state of the system 10 exists. Next, the patient line 50 is placed in fluid communication with the patient vein 60. At the same or substantially the same time, a blood pressure meter 80 is placed in fluid communication with the patient line 50 downstream of the pump 20 and flow meter 30. Pumping of the infusion fluid is started at the desired throughput and a user indicates to the controller 40 through the user interface 70 that a second calibration point is achieved. The control factors and pressures corresponding to the first and second calibration points are then correlated with the previously described experimentally obtained control factor/pressure data in order to derive real-time pressure data for the patient receiving infusion fluids from the pressure monitoring system 10.

Once the pressure monitoring system 10 has been calibrated as described above, the blood pressure meter 80 can be removed from the system 10 and the care giver can rely upon pressure monitoring system 10 to provide patient venous pressure P2. Accordingly, the patient can be relocated without having to sacrifice important patient information or without having to also relocate the costly blood pressure meter 80.

The controller 40 is operable to analyze and compare measured flow rate data with the desired flow rate input by the user. As necessary, the controller 40 may determine and communicate compensated or revised control factors to the pump 20 according to changing conditions, for example changes in patient venous pressure P2, encountered during infusion. The controller 40 is also operable to communicate any of the above described data to remote locations.

For example, when high calorie transfusions are received by a patient at the patient's home, the controller is operable to communicate the flow and determined patient venous pressure data to the patient's caregiver's hospital or clinic for monitoring. Should the controller 40 indicate to the caregiver that the patient's health is at risk or that a component of the infusion system requires attention, it is possible for the caregiver to rapidly assess the situation and dispatch patient assistance as required. Accordingly, the present invention is operable to reduce or prevent severe patient illness.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A method for monitoring patient circulatory pressure comprising:
generating a first infusion fluid pressure for delivery of an infusion fluid to a patient;
generating a second infusion fluid pressure responsive to a patient circulatory pressure;
determining patient circulatory pressure data based on a difference in control factors employed to generate the first infusion fluid pressure and the second infusion fluid pressure.

2. The method of claim 1 wherein the step of generating a first infusion fluid pressure for delivery of an infusion fluid to a patient comprises pumping infusion fluid through a piezoelectric driven pump.

3. The method of claim 1 wherein the step of generating a first infusion fluid pressure for delivery of an infusion fluid to a patient comprises determining an infusion fluid flow rate with a flow meter.

4. The method of claim 1 wherein the step of generating a first infusion fluid pressure for delivery of an infusion fluid to a patient comprises generating a first infusion fluid pressure greater than the patient circulatory pressure.

5. The method of claim 1 wherein the step of generating a second infusion fluid pressure responsive to a patient circulatory pressure comprises determining an infusion fluid flow rate with a flow meter.

6. The method of claim 1 wherein the step of generating a second infusion fluid pressure responsive to a patient circulatory pressure comprises generating a second infusion fluid pressure greater than the patient circulatory pressure.

7. The method of claim 1 wherein the step of generating a second infusion fluid pressure responsive to a patient circulatory pressure comprises providing a control factor to an infusion pump.

8. The method of claim 7 wherein the step of providing a control factor to an infusion pump comprises determining the control factor based upon infusion fluid flow data received from a flow meter.

10. The method of claim 1 wherein the step of determining patient circulatory pressure data based on a difference in control factors employed to generate the first infusion fluid pressure and the second infusion fluid pressure comprises analyzing control factors employed to direct operation of an infusion pump.

11. The method of claim 1 further comprising the step of correlating specific control factors to a range of different pressures.

12. The method of claim 1 further comprising the step of designating control factors for an infusion pressure equal to zero.

13. A patient circulatory pressure monitoring system comprising:
an infusion pump;
a flow meter in fluid communication with the infusion pump; and
a controller in electrical communication with the infusion pump and the flow meter, the controller further comprising a data correlation of infusion pump control factors and circulatory pressures.

14. The system of claim 13 wherein the infusion pump is a piezoelectric driven infusion pump.

15. The system of claim 13 wherein a control factor comprises a voltage.

16. The system of claim 13 wherein a control factor comprises a frequency.

17. A method for monitoring patient circulatory pressure comprising:
providing a first control factor to an infusion pump;
providing a second control factor to an infusion pump responsive to a patient circulatory pressure;
determining patient circulatory pressure data based on a difference in the first control factor and second control factor.

18. The method of claim 17 wherein the step of providing a first control factor to an infusion pump comprises providing a first control factor to a piezoelectric driven infusion pump.

19. The method of claim 17 wherein the step of providing a first control factor to an infusion pump comprises directing the infusion pump to generate an infusion fluid pressure greater than the patient circulatory pressure.

20. The method of claim 17 wherein the step of providing a second control factor to an infusion pump responsive to a patient circulatory pressure comprises directing the infusion pump to generate an infusion fluid pressure greater than the patient circulatory pressure
